(19) United States
(12) Patent Application Publication
(10) Pub. No.: US 2026/0216026 A1
(43) Pub. Date: Jul. 30, 2026

(54) COSMETIC COMPOSITION

(71) Applicant: SHISEIDO COMPANY, LTD., Chuo-ku, Tokyo (JP)

(72) Inventors: Kazutaka KANAYAMA, Tokyo (JP); Mizuki YAMAMOTO, Tokyo (JP); Akari MIZUGUCHI, Tokyo (JP)

(73) Assignee: SHISEIDO COMPANY, LTD., Chuo-ku, Tokyo (JP)

(21) Appl. No.: 19/144,750

(22) PCT Filed: Jan. 17, 2024
(86) PCT No.: PCT/JP2024/001106

§ 371 Date: Jun. 30, 2025

(30) Foreign Application Priority Data
Jan. 31, 2023 (JP) 2023-012909

(51) Int. Cl. A61K 8/42; A61K 8/37; A61Q 1/06; A61Q 1/08; A61Q 1/10; A61Q 17/04; A61Q 19/02

(52) U.S. Cl. CPC A61K 8/42; A61K 8/375; A61Q 1/06; A61Q 1/08; A61Q 1/10; A61Q 17/04; A61Q 19/02; A61K 2800/43; A61K 2800/612

(57) ABSTRACT

Provided is a cosmetic composition in which dispersibility of a powder, surface-treated with an amide compound and/or a salt thereof or a metal soap, in a polar oil is improved. A composition that is a cosmetic composition, comprising a component (A): an inorganic pigment surface-treated with the following (i) or (ii): (i) an amide compound obtained by dehydration condensation of an acidic amino acid and a carboxylic acid, and/or a salt thereof, or (ii) a metal soap; a component (B): a (polyglyceryl-2 isostearate/dimer dilinoleate) copolymer; and a component (C): a liquid oil comprising a polar oil, wherein an apparent IOB value of the component (C) is 0.09 or greater and 0.37 or less.

### FIELD

The present invention relates to a cosmetic composition.

### BACKGROUND

Makeup cosmetics, such as cosmetic primers, foundations, powders, blushes, eyeshadows, lipsticks, eyeliners, and eyebrow products, and skin care cosmetics, such as whitening cosmetics and UV care cosmetics, are known as cosmetics that contain inorganic pigments.

Inorganic pigments and oils are often blended in such cosmetics. However, powders that have not been surface-treated, particularly powders that contain metal elements, can act as catalysts in cosmetics and degrade the components of the cosmetics. Therefore, there is a need for surface-treated inorganic pigments.

As a surface-treated inorganic pigment, PTL 1 discloses a hydrophilically treated powder, surface-treated with a hydrolyzable silyl group-containing polyether modified silicone represented by general formula (1) (R¹ₐR²_{b}R³_{c}SiO_{(4-a-b-c)/2)}.

PTL 2, for example, discloses cosmetics in which a (polyglyceryl-2 isostearate/dimer dilinoleate) copolymer is blended, such as “Lipstick formulation 1” described in paragraph 0121, “Foundation formulation” described in paragraph 0127, and “Eyeshadow formulation” described in paragraph 0129.

### CITATION LIST

### Patent Literature

[PTL 1] Japanese Unexamined Patent Publication (Kokai) No. 2004-155978
[PTL 2] WO 2015/108176

### SUMMARY

### Technical Problem

Cosmetics in which naturally derived components are blended are preferred. In makeup products, many pigments are blended therein for the aesthetic effect thereof, and attempts have been made to stabilize inorganic pigments such as metal oxides by treating surfaces with lipophilic substances. In addition, as naturally derived components, there are amino acid-treated pigments, which utilize amino acids amidated with carboxylic acids, and metal soap-treated pigments, which are composed of higher fatty acids and alkaline earth metals.

The present inventors have found that these pigments that were subjected to surface treatments tend to have poor dispersibility in polar oils commonly used in cosmetics. When agglomeration of a surface-treated pigment occurs, the appearance of the cosmetic darkens, and the beauty thereof is impaired.

The present invention aims to improve the above circumstances, and an object thereof is to provide a cosmetic composition in which dispersibility of an inorganic pigment, surface-treated with an amide compound and/or a salt thereof or a metal soap, in a polar oil is improved.

### Solution to Problem

The present invention that achieves the above object is as follows.

### <Aspect 1>

A composition that is a cosmetic composition, comprising
a component (A): an inorganic pigment surface-treated with (i) or (ii) below:
   (i) an amide compound obtained by dehydration condensation of an acidic amino acid and a carboxylic acid, and/or a salt thereof, or
   (ii) a metal soap;
a component (B): a (polyglyceryl-2 isostearate/dimer dilinoleate) copolymer; and
a component (C): a liquid oil comprising a polar oil,
wherein an apparent IOB value of the component (C) is 0.09 or greater and 0.37 or less.

### <Aspect 2>

The composition according to Aspect 1,
wherein with respect to the (i), number of carbon atoms in the carboxylic acid is 18 or less, and/or,
wherein with respect to the (ii), number of carbon atoms in a fatty acid forming the metal soap is 12 or greater.

### <Aspect 3>

The composition according to Aspect 1 or 2, wherein an IOB value of the polar oil is 0.10 or greater and 0.55 or less.

### <Aspect 4>

The composition according to any one of Aspects 1 to 3, wherein a content of the component (B) is 24% by mass or less.

### <Aspect 5>

The composition according to any one of Aspects 1 to 4, wherein a ratio of a content of the component (B) to a content of the component (A) (a content of the component (B)/a content of the component (A)) is 0.08 or greater and 2.0 or less.

### <Aspect 6>

The composition according to any one of Aspects 1 to 5, wherein, when further comprising a silicone oil, a content of the silicone oil is 50% by mass or less, and the apparent IOB value of the component (C) is calculated by excluding the silicone oil.

### <Aspect 7>

The composition according to any one of Aspects 1 to 6, wherein the polar oil comprises at least one selected from a group consisting of triisostearin, polyglyceryl-2 triisostearate, pentaerythrityl tetraethylhexanoate, neopentyl glycol dicaprate, glyceryl diisostearate, diisostearyl malate, and trimethylolpropane triethylhexanoate.

### <Aspect 8>

The composition according to any one of Aspects 1 to 7, wherein a content of the polar oil is 50% by mass or greater.

### Advantageous Effects of Invention

According to the present invention, in a polar oil, dispersibility of an inorganic pigment treated with an amide compound and/or a salt thereof or a metal soap is improved.

### DESCRIPTION OF EMBODIMENTS

Hereinafter, embodiments of the present invention will be described in detail. Note that the present invention is not limited to the following embodiments, and various modifications can be made within the scope of the invention.

### <<Cosmetic Composition>>

The cosmetic composition of the present invention (hereinafter, also simply referred to as “composition of the present invention”) is
a composition comprising
a component (A): an inorganic pigment surface-treated with the following (i) or (ii):
   (i) an amide compound obtained by dehydration condensation of an acidic amino acid and a carboxylic acid, and/or a salt thereof, or
   (ii) a metal soap;
a component (B): a (polyglyceryl-2 isostearate/dimer dilinoleate) copolymer; and
a component (C): a liquid oil comprising a polar oil,
wherein an apparent IOB value of the component (C) is 0.09 or greater and 0.37 or less.

According to the intensive studies by the present inventors, it was discovered that in a polar oil, when a (polyglyceryl-2 isostearate/dimer dilinoleate) copolymer is blended together with the inorganic pigment surface-treated with (i) or (ii) described above, dispersibility of the inorganic pigment surface-treated with (i) or (ii) described above is significantly improved. Without wishing to be bound by theory, the mechanism thereof is described below.

The inorganic pigment surface-treated with (i) or (ii) described above still has a portion on the surface thereof where hydrophilic groups are exposed, i.e., a hydrophilic surface is exposed. Therefore, there is a problem that in an oil phase, particularly a liquid oil comprising a polar oil, dispersibility of the inorganic pigment surface-treated with (i) or (ii) described above is poor. In contrast, by blending a (polyglyceryl-2 isostearate/dimer dilinoleate) copolymer in the oil phase, the polyglyceryl-2 portion of the (polyglyceryl-2 isostearate/dimer dilinoleate) copolymer is adsorbed onto the exposed hydrophilic surface of the inorganic pigment surface-treated with (i) or (ii) described above, while a structure in which the dimer dilinoleate backbone portion is oriented towards the polar oil is formed. Since the dimer dilinoleate has a high affinity for polar oils, it is presumed that such a structure improves dispersibility of the inorganic pigment surface-treated with (i) or (ii) described above in an oil phase, particularly in a polar oil. In other words, it is presumed that the (polyglyceryl-2 isostearate/dimer dilinoleate) copolymer fulfills a role of a dispersant of the inorganic pigment surface-treated with (i) or (ii) described above in an oil phase (particularly, an oil comprising a polar oil).

In the composition of the present invention, the dispersibility of the inorganic pigment can be evaluated by the lightness of a prepared composition. Based on the finding that the smaller the particle size of the inorganic pigment particles, the more white light is reflected from the surface, and thus the greater the lightness of the entire composition, it is suggested that the greater the lightness of the composition of the present invention, the better the dispersibility of the surface-treated inorganic pigment contained in the composition of the present invention. More specifically, for example, by setting a composition in which the (polyglyceryl-2 isostearate/dimer dilinoleate) copolymer is not blended as a reference color (L0), and measuring L1 as the lightness of the composition of the present invention in which the (polyglyceryl-2 isostearate/dimer dilinoleate) copolymer is blended, it can be said that the larger the lightness difference (ΔL=L1−L0) therebetween, the more improved the dispersibility of the surface-treated inorganic pigment in the composition of the present invention. Thus, the composition of the present invention may have a lightness difference (ΔL) from the reference color of, for example, 0.05 or greater, 0.10 or greater, 0.20 or greater, 0.30 or greater, 0.40 or greater, 0.50 or greater, 0.60 or greater, 0.70 or greater, 0.8 or greater, 0.90 or greater, 1.00 or greater, 1.50 or greater, or 2.00 or greater. Note that lightness (L value) may be measured by any colorimeter.

Incidentally, several cosmetic compositions in which a (polyglyceryl-2 isostearate/dimer dilinoleate) copolymer is blended in an oil phase have been reported (for example, PTL 2). The (polyglyceryl-2 isostearate/dimer dilinoleate) copolymer in this case is considered to be used as a polar oil, not for the purpose of using as a dispersant of an inorganic pigment.

Therefore, the present invention, as described below, also provides a use of the (polyglyceryl-2 isostearate/dimer dilinoleate) copolymer as a dispersant of an inorganic pigment.

Hereinafter, details of the composition of the present invention will be described.

### <Component (A)>

The composition of the present invention comprises, as the component (A), an inorganic pigment surface-treated with the following (i) or (ii):
(i) an amide compound obtained by dehydration condensation of an acidic amino acid and a carboxylic acid, and/or a salt thereof, or
(ii) a metal soap.

In the present invention, the inorganic pigment surface-treated with the above (i) or (ii) is dispersed in an oil comprising a polar oil due to the presence of a (polyglyceryl-2 isostearate/dimer dilinoleate) copolymer.

Inorganic pigments before surface treatment include, but are not limited to, pigment particles (particularly, coloring pigment particles) and ultraviolet-scattering particles. An inorganic pigment before surface treatment may be a powder containing a metal element.

In the present invention, the pigment particle (particularly, coloring pigment particle) is not particularly limited, and includes pigment particles generally used in makeup cosmetics. More specifically, examples of the pigment particle include inorganic pigments such as talc, mica, kaolin, silica, calcium carbonate, zinc oxide, titanium oxide (titanium dioxide), red iron oxide, yellow iron oxide, black iron oxide, ultramarine, Prussian blue, carbon black, low-order titanium oxide, cobalt violet, chromium oxide, chromium hydroxide, cobalt titanate, bismuth oxychloride, and titanium-mica-based pearl pigments; organic pigments such as zirconium, barium, and aluminum lakes, such as Red No. 201, Red No. 202, Red No. 204, Red No. 205, Red No. 220, Red No. 226, Red No. 228, Red No. 405, Orange No. 203, Yellow No. 205, Yellow No. 4, Yellow No. 5, Blue No. 1, Blue No. 404, and Green No. 3; natural colors such as chlorophyll and β-carotene; and dyes. In addition, the average primary particle size of the pigment particles is not particularly limited and, for example, may be 0.02 μm or more and 1.0 μm or less. Note that in the present invention, average primary particle size can be measured by transmission electron microscope images.

In the present invention, the ultraviolet-scattering particle is not particularly limited, and includes ultraviolet-scattering particles generally used in makeup cosmetics. More specifically, examples of the ultraviolet-scattering particle include metal oxides selected from one or more of titanium oxide, zinc oxide, and cerium oxide. In addition, the average primary particle size of the ultraviolet-scattering particles is not particularly limited and, for example, may be 10 nm or more and 200 nm or less.

(i) Amide Compound Obtained by Dehydration Condensation of Acidic Amino Acid and Carboxylic Acid, and/or Salt Thereof

In the present invention, an acidic amino acid refers to an amino acid having a larger number of carboxyl groups than number of amino groups. More specifically, examples of the acidic amino acid include, but are not limited to, glutamic acid and aspartic acid. Among these, glutamic acid is more preferable. An amide compound obtained by dehydration condensation of an acidic amino acid and a carboxylic acid refers to a compound in which the amino group of the amino acid undergoes dehydration condensation with a carboxylic acid to form a new amide bond. The number of carbon atoms in the carboxylic acid for forming an amide bond with an acidic amino acid is not particularly limited and, for example, may be one or greater, two or greater, 3 or greater, 4 or greater, 5 or greater, or 12 or greater, and may be 18 or less, 17 or less, or 16 or less. Specific examples of the carboxylic acid are not particularly limited, and examples include stearic acid. In addition, a salt of an amide compound obtained by dehydration condensation of an acidic amino acid and a carboxylic acid is not particularly limited and, for example, may be a metal salt of sodium or potassium.

In the present invention, the “inorganic pigment surface-treated with (i) an amide compound obtained by dehydration condensation of an acidic amino acid and a carboxylic acid, and/or a salt thereof” may include pigment particles surface-treated with an ester-based surface treatment agent comprising an amino acid, such as an ester-amino acid. The ester-based surface treatment agent comprising an amino acid is not particularly limited, and examples include isostearyl sebacate-disodium stearoyl glutamate. Further, in the present invention, a condensate such as sodium dilauroyl glutamate lysine, in which two sodium lauroyl glutamate are linked by lysine, a type of basic amino acid, may be used. Since sodium lauroyl glutamate corresponds to “(i) an amide compound obtained by dehydration condensation of an acidic amino acid and a carboxylic acid, and/or a salt thereof”, sodium dilauroyl glutamate lysine is also considered to correspond to (i).

### (ii) Metal Soap

A metal soap for treating surfaces of an inorganic pigment is also referred to as a “metal salt of a fatty acid”. The fatty acid forming the metal soap, for example, is preferably a fatty acid having 12 or more carbon atoms. The upper limit of the number of carbon atoms of the fatty acid is not particularly limited and, for example, may be 32 or less, 24 or less, or 18 or less. In addition, the metal salt of a fatty acid is not particularly limited and, for example, may be a salt of calcium, aluminum, or magnesium. More specifically, in the present invention, the metal soap is not particularly limited, and examples include calcium stearate, aluminum dimyristate, and magnesium stearate.

In the present invention, a commercially available inorganic pigment surface-treated with the above (i) or (ii) may be used, or the inorganic pigment before surface treatment described above that is treated by a known surface treatment method, for example, a method such as a dry treatment or a wet treatment, using an appropriate treatment agent that is (i) an amide compound obtained by dehydration condensation of an acidic amino acid and a carboxylic acid, and/or a salt thereof, or (ii) a metal soap, may be used.

In the present invention, the content of the component (A) is not particularly limited and, for example, may be 30% by mass or less, 28% by mass or less, 26% by mass or less, 24% by mass or less, 22% by mass or less, 20% by mass or less, 18% by mass or less, 16% by mass or less, 14% by mass or less, or 12% by mass or less, and may be 0.001% by mass or greater, 0.01% by mass or greater, 0.1% by mass or greater, 1.0% by mass or greater, 10% by mass or greater, or 12% by mass or greater, relative to the entire cosmetic composition.

In the present invention, the inorganic pigment surface-treated with the above (i) or (ii), before being blended in an oil, may be a surface-treated inorganic pigment dispersion that has been dispersed in advance by mechanical force. The “mechanical force” is not particularly limited, and examples include mixing with a roller. In addition, the dispersion medium in this case is not particularly limited and, for example, may be the polar oil described below. Further, the ratio of the inorganic pigment surface-treated with the above (i) or (ii) to the dispersion medium thereof in this case is not particularly limited, and the inorganic pigment surface-treated with the above (i) or (ii): the dispersion medium thereof (mass ratio), for example, may be 1:9 to 9:1, 2:8 to 8:2, 2.5:7.5 to 7.5:2.5, 3:7 to 7:3, 3.5:6.5 to 6.5:3.5, 4:6 to 6:4, or 5:5.

### <Component (B)>

The composition of the present invention comprises a (polyglyceryl-2 isostearate/dimer dilinoleate) copolymer as the component (B).

The (polyglyceryl-2 isostearate/dimer dilinoleate) copolymer can be generated by condensation reaction of diglycerin, isostearic acid, and hydrogenated dimer dilinoleate. One type of (polyglyceryl-2 isostearate/dimer dilinoleate) copolymer used in the present invention may be, for example, one that is commercially available from Kokyu Alcohol Kogyo Co. of Japan under the trade name “HAILUCENT ISDA”.

In the present invention, the content of the component (B) ((polyglyceryl-2 isostearate/dimer dilinoleate) copolymer) is not particularly limited and, for example, may be 0.01% by mass or greater, 0.02% by mass or greater, 0.03% by mass or greater, 0.04% by mass or greater, 0.05% by mass or greater, 0.06% by mass or greater, 0.07% by mass or greater, 0.08% by mass or greater, 0.09% by mass or greater, 0.1% by mass or greater, 0.2% by mass or greater, 0.3% by mass or greater, 0.4% by mass or greater, 0.5% by mass or greater, 0.6% by mass or greater, 0.7% by mass or greater, 0.8% by mass or greater, 0.9% by mass or greater, 1.0% by mass or greater, 5.0% by mass or greater, 8.0% by mass or greater, 10% by mass or greater, 15% by mass or greater, or 20% by mass or greater, and may be 24% by mass or less, 22% by mass or less, 20% by mass or less, 18% by mass or less, 15% by mass or less, or 12% by mass or less, relative to the entire composition.

In the present invention, the ratio of the content of the component (B) to the content of the component (A) described above (content of component (A)/content of component (B)) is not particularly limited and, for example, may be 0.08 or greater, 0.10 or greater, 0.20 or greater, 0.30 or greater, 0.40 or greater, 0.50 or greater, 0.60 or greater, 0.70 or greater, 0.80 or greater, 0.90 or greater, 1.0 or greater, or 1.5 or greater, and may be 2.0 or less, 1.8 or less, 1.5 or less, or 1.0 or less.

### <Component (C)>

The composition of the present invention comprises, as the component (C), a liquid oil. The liquid oil refers to an oil in a liquid state at room temperature (25° C.). In the present invention, the liquid oil comprises a polar oil as an indispensable component. In addition, the liquid component may optionally comprise a hydrocarbon oil and/or a silicone oil.

In the present invention, the apparent IOB value of the component (C) is 0.09 or greater and 0.37 or less. The apparent IOB value, more specifically, may be 0.09 or greater, 0.10 or greater, 0.11 or greater, 0.12 or greater, 0.13 or greater, 0.14 or greater, 0.15 or greater, 0.16 or greater, 0.17 or greater, 0.18 or greater, 0.19 or greater, 0.20 or greater, 0.21 or greater, 0.22 or greater, 0.23 or greater, 0.24 or greater, 0.25 or greater, 0.26 or greater, 0.27 or greater, 0.28 or greater, 0.29 or greater, or 0.30 or greater, and may be 0.37 or less, 0.36 or less, 0.35 or less, 0.34 or less, 0.33 or less, 0.32 or less, 0.31 or less, 0.30 or less, 0.29 or less, 0.28 or less, 0.27 or less, 0.26 or less, 0.25 or less, 0.24 or less, 0.23 or less, 0.22 or less, 0.21 or less, or 0.20 or less. Note that IOB value is an abbreviation for inorganic/organic balance, is a value that represents a ratio of inorganic value to organic value, and acts as an indicator showing the degree of polarity of an organic compound. The IOB value, specifically, is represented as IOB value=inorganic value/organic value. With respect to each of “inorganic value” and “organic value”, the “inorganic value” and “organic value” are set according to various atoms or functional groups, such that, for example, the “organic value” for one carbon atom in a molecule is 20, and the “inorganic value” for one hydroxyl group is 100. The IOB value of an organic compound can be calculated by summing the “inorganic values” and “organic values” of all atoms and functional groups in the organic compound (refer to, for example, Yoshio Koda, “Organic Conceptual Diagrams—Fundamentals and Applications—”, pp. 11 to 17, Sankyo Publishing Co., Ltd., published in 1984).

In the composition of the present invention, the apparent IOB value of the component (C), if comprising only one oil as the liquid oil, is a value obtained by dividing the product of the IOB value of the oil and the mass percentage of the oil relative to the entire composition by the mass percentage of the oil. Specifically, for example, if only an oil A is contained as the liquid oil, when the IOB value of the oil A is defined as “a” and the content ratio of the oil A relative to the entire composition is defined as “x % by mass”, the “apparent IOB value of component (C)” in this composition can be determined from the following formula (1):

$$Apparent IOB value of component ( C ) = a \qquad ( 1 )$$

In the composition of the present invention, the apparent IOB value of the component (C), if comprising two or more oils as the liquid oil, is a value obtained by dividing the product of IOB value of each oil and the weight percentage of each oil in the entire composition by the mass percentage of all oils. Specifically, for example, if two different oils A and B are contained as the liquid oil, when the IOB value of the oil A is defined as “a”, the content ratio of the oil A relative to the entire cosmetic is defined as “x % by mass”, the IOB value of the oil B is defined as “b”, and the content ratio of the oil B relative to the entire cosmetic is defined as “y % by mass”, the “apparent IOB value of component (C)” in this composition can be determined from the following formula (2):

$$Apparent IOB value of component ( C ) = ( a \times x \% + b \times y \% ) / ( x \% + y \% ) \qquad ( 2 )$$

In the present invention, the apparent IOB value of the component (C) is calculated by excluding the component (B) described above and, if a silicone oil is contained, the silicone oil.

### (Polar Oil)

In the present invention, the “polar oil” refers to all oils having a polarity, among oils that can be used in cosmetics, other than the (polyglyceryl-2 isostearate/dimer dilinoleate) copolymer. Saturated hydrocarbon oils (IOB value=0) and silicone oils (IOB cannot be defined) naturally do not correspond to polar oils. More specifically, in the present invention, the polar oil, for example, may have an IOB value of 0.10 or greater, 0.11 or greater, 0.12 or greater, or 0.13 or greater. In addition, in the present invention, the upper limit of the IOB value of the polar oil is not particularly limited and, for example, may be 0.55 or less, 0.52 or less, 0.50 or less, 0.45 or less, or 0.40 or less.

In the present invention, the polar oil may be at least one selected from the group consisting of ester oils, ether oils, higher alcohols, and fatty acids. More specifically, the polar oil is not particularly limited and, for example, may comprise at least one selected from the group consisting of polyglyceryl-10 laurate (IOB value=1.60), PEG-20 sorbitan cocoate (IOB value=1.49), bis-ethoxydiglycol cyclohexane-1,4-dicarboxylate (IOB value=1.03), phenoxyethyl caprylate (IOB value=0.29), PPG-30-buteth-30 (IOB value=0.94), diisopropyl sebacate (IOB value=0.4), neopentyl glycol diheptanoate (IOB value=0.33), diethylhexyl succinate (IOB value=0.32), isononyl isononanoate (IOB value=0.2), isopropyl myristate (IOB value=0.18), isoamyl laurate (IOB value=0.18), PPG-2 myristyl propionate (IOB value=0.26), polyglyceryl-2 triisostearate (IOB value=0.26), polyglyceryl-2 diisostearate (IOB value=0.41), diethoxyethyl succinate (IOB value=1.13), dioctyl adipate (IOB value=0.29), diisopropyl adipate (IOB value=0.55), dioctyl sebacate (IOB value=0.24), di-PPG-3 myristyl adipate (IOB value=0.55), PPG-14 butyl (IOB value=0.41), PPG-15 stearyl (IOB value=0.32), PPG-3 myristyl (IOB value=0.35), octyl palmitate (IOB value=0.13), isopropyl palmitate (IOB value=0.16), butyl stearate (IOB value=0.14), hexyl laurate (IOB value=0.17), myristyl myristate (IOB value=0.11), decyl oleate (IOB value=0.11), isotridecyl isononanoate (IOB value=0.15), cetyl ethylhexanoate (IOB value=0.13), pentaerythrityl tetraethyhexanoate (IOB value=0.35), dioctyl succinate (IOB value=0.36), glycol distearate (IOB value=0.16), glyceryl diisostearate (IOB value=0.29), neopentyl glycol dicaprate (IOB value=0.25), diisostearyl malate (IOB value=0.28), trimethylolpropane triethyhexanoate (IOB value=0.33), trimethylolpropane triisostearate (IOB value=0.16), glyceryl tri-2-ethylhexanoate (triethylhexanoin) (IOB value=0.35), trimethylolpropane trioctanoate (IOB value=0.33), trimethylolpropane triisostearate (IOB value=0.16), diisobutyl adipate (IOB value=0.46), N-lauroyl-L-glutamic acid-2-octyldodecyl ester (IOB value=0.29), 2-hexyldecyl adipate (IOB value=0.16), ethylhexyl methoxycinnamate (IOB value=0.28), 2-ethylhexyl palmitate (IOB value=0.13), 2-ethylhexyl ethylhexanoate (IOB value=0.2), triisostearin (IOB value=0.16), PPG-3 dipivalate (IOB value=0.52), glyceryl tri (caprylate/caprate) (IOB value=0.33), alkyl benzoate (C₁₂₋₁₅) (IOB value=0.18), phenethyl benzoate (IOB value=0.3), butyloctyl salicylate (IOB value=0.43), and neopentyl glycol diheptanoate (IOB value=0.33). Among these, it is particularly preferable that the polar oil comprise at least one of triisostearin, polyglyceryl-2 triisostearate, pentaerythrityl tetraethylhexanoate, neopentyl glycol dicaprate, glyceryl diisostearate, diisostearyl malate, and trimethylolpropane triethylhexanoate.

Examples of ultraviolet absorbers that can be considered as polar oils include ultraviolet absorbers having an IOB of 0.10 or greater. Specifically, examples include, but are not limited to, organic ultraviolet absorbers such as ethylhexyl methoxycinnamate, octocrylene, polysilicone-15, t-butyl methoxydibenzoylmethane, ethylhexyl triazone, bis-ethylhexyloxyphenol methoxyphenyl triazine, diethylamino hydroxybenzoyl hexyl benzoate, oxybenzone-3, methylene bis-benzotriazolyl tetramethylbutylphenol, homosalate, and ethylhexyl salicylate. These ultraviolet absorbers can be used independently or in combinations of two or more.

In the composition of the present invention, the content of the polar oil is not particularly limited and, for example, may be 45% by mass or greater, 50% by mass or greater, 55% by mass or greater, 60% by mass or greater, 65% by mass or greater, 70% by mass or greater, 75% by mass or greater, or 80% by mass or greater, and may be 90% by mass or less, 85% by mass or less, or 80% by mass or less, relative to the entire composition.

In the present invention, the content of the polar oil in the oil is not particularly limited and, for example, may be 10% by mass or greater, 15% by mass or greater, 20% by mass or greater, 25% by mass or greater, 30% by mass or greater, 35% by mass or greater, 40% by mass or greater, 45% by mass or greater, 50% by mass or greater, or 55% by mass or greater, and may be 99% by mass or less, 95% by mass or less, 90% by mass or less, 85% by mass or less, 80% by mass or less, 75% by mass or less, 70% by mass or less, 65% by mass or less, or 60% by mass or less, relative to the entire oil.

### (Hydrocarbon Oil)

The composition of the present invention may further comprise a hydrocarbon oil as an optional liquid oil in the component (C). Note that the IOB value of the hydrocarbon oil is considered to be 0 (zero).

Specific examples of the hydrocarbon oil are not particularly limited, and examples include olefin oligomers, squalane, liquid paraffin, light isoparaffin, heavy liquid isoparaffin, and hydrogenated polydecene.

In the composition of the present invention, the content of the hydrocarbon oil, if comprising any, is not particularly limited and, for example, may be 0.5% by mass or greater, 1.0% by mass or greater, 5.0% by mass or greater, 10% by mass or greater, 15% by mass or greater, or 20% by mass or greater, and may be 50% by mass or less, 45% by mass or less, 40% by mass or less, 35% by mass or less, or 30% by mass or less, relative to the entire composition.

### (Silicone Oil)

The composition of the present invention may further comprise a silicone oil as an optional liquid oil in the component (C). As described above, the apparent IOB value of the component (C) is calculated by excluding the silicone oil.

Specific examples of the silicone oil are not particularly limited, and examples include, but are not limited to, dimethyl silicone, phenylmethyl silicone, cyclomethicone, aminopropyl dimethicone, (alkyl acrylate/dimethicone) copolymer, (acrylates/ethylhexyl acrylate/dimethicone methacrylate) copolymer, (acrylates/stearyl acrylate/dimethicone methacrylate) copolymer, (acrylates/tridecyl acrylate/triethoxysilylpropyl methacrylate/dimethicone methacrylate) copolymer, (acrylates/behenyl acrylate/dimethicone methacrylate) copolymer, (acrylates/dimethicone) copolymer, PEGylated dimethicone, and diphenylsiloxyphenyl trimethicone.

In the composition of the present invention, the content of the silicone oil, if comprising any, is not particularly limited and, for example, may be 0.5% by mass or greater, 1.0% by mass or greater, 5.0% by mass or greater, or 10% by mass or greater, relative to the entire composition. The upper limit of the content of the silicone oil, if comprising any, is preferably 50% by mass or less, and more preferably 40% by mass or less, 30% by mass or less, 20% by mass or less, 10% by mass or less, or 5.0% by mass or less.

### <Additional Component>

The composition of the present invention may further optionally comprise an additional component, as long as the effect of the present invention is not impaired. The additional component is not particularly limited, include and examples waxes (for example, polyethylene/microcrystalline wax) that are solid at room temperature.

The content of the additional component of the present invention, if further comprising any, is not particularly limited and, for example, may be 0.01% by mass or greater, 0.1% by mass or greater, 0.5% by mass or greater, 1.0% by mass or greater, or 5.0% by mass or greater, and may be 20% by mass or less, 10% by mass or less, or 5.0% by mass or less, relative to the entire composition.

### (Dosage Form and Application of Composition of Present Invention)

The composition of the present invention is not particularly limited in the dosage form thereof. More specifically, the cosmetic of the present invention may be, for example, an oily cosmetic or an oil-in-water type or water-in-oil type emulsified cosmetic. An oil phase comprising an oil relating to the composition of the present invention may be a continuous phase, or may be a dispersed phase. In addition, the composition of the present invention may be further blended with water, an aqueous component, or another component in accordance with the intended dosage form.

Examples of applications of the composition of the present invention include, but are not limited to, makeup cosmetics such as cosmetic primers, liquid foundations, liquid blushes, liquid eyeshadows, and liquid foundation lipsticks; and skin care cosmetics such as toners, emulsions, whitening cosmetics, and UV care cosmetics. Water, an aqueous component, or another component may be appropriately blended in the composition of the present invention described above in accordance with the intended application.

### <<Application of (Polyglyceryl-2 Isostearate/Dimer Dilinoleate) Copolymer>>

The present invention also provides a use of the (polyglyceryl-2 isostearate/dimer dilinoleate) copolymer as a dispersant of an inorganic pigment.

The inorganic pigment is not particularly limited, but from the viewpoint of notably exhibiting the dispersing effect of the (polyglyceryl-2 isostearate/dimer dilinoleate) copolymer, is preferably an inorganic pigment surface-treated with the following (i) or (ii):
(i) an amide compound obtained by dehydration condensation of an acidic amino acid and a carboxylic acid, and/or a salt thereof, or
(ii) a metal soap.

Note that details of the “inorganic pigment surface-treated with (i) or (ii)” are described above.

### EXAMPLES

The present invention will be further described in detail with reference to the Examples below. However, the present invention is not limited thereto.

### Examples 1 to 3 and Comparative Example 1

Based on the formulations in Table 1, the compositions of Examples 1 to 3 and Comparative Example 1 were each prepared. Note that the numerical values up to the row “Total” in Table 1 and Table 2 to Table 7 described below indicate the blending amount (content) of each constituent component, and the unit is % by mass.

To check dispersibility of the component (A) in each of the prepared compositions, lightness value (L value) of each thereof was measured with a colorimeter (the same apparatus and the same conditions were used in each of the following experiments). More specifically, each sample was placed in 100 mL of PP and then heated to 90 to 95° C. to be dissolved. The mixture was stirred for 120 seconds with a Mazerustar. The mixture was then reheated to 90 to 95° C., and a metal plate was filled therewith to measure the L value.

The lightness of the composition of Comparative Example 1 was set as the reference color, and the lightness difference (ΔL value=L value of composition of each Example−L value of reference color) between each composition of Examples 1 to 3 and the reference color was determined. Results are shown in Table 1. Note that in Tables 1 to 7, in the results of “Lightness difference (ΔL)”, “L” and “D” are symbols that represent the direction of a vector connecting two points: the reference color and the color of another Example or Comparative Example. In this case, on the measuring device, if the color difference was positive, “L” was displayed, and if the color difference was negative, “D” was displayed.

TABLE 1
(Table 1 Examples 1 to 3 and Comparative Example 1)

| | | Comparative | Example | Example | Example |
|---|---|---|---|---|---|
| Constituent component | | Example 1 | 1 | 2 | 3 |
| Additional | Polyethylene/microcrystalline | 8.0 | 8.0 | 8.0 | 8.0 |
| component | wax | | | | |
| Component | Yellow iron oxide treated | 12 | 12 | 12 | 12 |
| (A) | with isostearyl sebacate, | | | | |
| | disodium stearoyl glutamate, | | | | |
| | and aluminum hydroxide | | | | |
| Component | (Polyglyceryl-2 isostearate/ | — | 24 | 1.0 | 12 |
| (B) | dimer dilinoleate) copolymer | | | | |
| Component | Triisostearin [polar oil] | 80 | 56 | 79 | 68 |
| (C) | (IOB value = 0.16) | | | | |

| | | | | |
|---|---|---|---|---|
| Total | 100 | 100 | 100 | 100 |
| Apparent IOB value of component (C) | 0.16 | 0.16 | 0.16 | 0.16 |
| Content of component (B)/ | 0 | 2 | 0.08333 | 1 |
| content of component (A) | | | | |
| Lightness difference (ΔL) | Reference | 2.22 L | 1.38 L | 2.16 L |
| | color | | | |

As is clear from the results in Table 1, it was found that the compositions of Examples 1 to 3 all had improved dispersibility of the component (A) compared to Comparative Example 1.

### Examples 4 to 8 and Comparative Examples 2 to 4

Based on the formulations in Table 2, the compositions of Examples 4 to 8 and Comparative Examples 2 to 4 were each prepared. The lightness value (L value) of each of the prepared compositions was measured, and the lightness difference from a reference color was determined. Results are shown in Table 2. Note that for Examples 4 to 8, Comparative Example 2 was used as the reference color. For Comparative Examples 3 and 4, as reference colors, those obtained by removing the component (B) from each of Comparative Examples 3 and 4 were used.

TABLE 2
(Table 2 Examples 4 to 8 and Comparative Examples 2 to 4)

| | | | Comparative | Example | Example | Example |
|---|---|---|---|---|---|---|
| Constituent component | | | Example 2 | 4 | 5 | 6 |
| Additional component | | Polyethylene/ | 8.0 | 8.0 | 8.0 | 8.0 |
| | | microcrystalline wax | | | | |
| Component (A) | | Yellow iron oxide treated with | 12 | 12 | 12 | 12 |
| | | isostearyl sebacate, | | | | |
| | | disodium stearoyl glutamate, | | | | |
| | | and aluminum hydroxide | | | | |
| Component (B) | | (Polyglyceryl-2 isostearate/ | — | 1.0 | 1.0 | 1.0 |
| | | dimer dilinoleate) copolymer | | | | |
| Component | Polar oil | Polyglyceryl-2 triisostearate | 80 | 79 | — | — |
| (C) | | (IOB value = 0.26) | | | | |
| | | Pentaerythrityl | — | — | 79 | — |
| | | tetraethylhexanoate | | | | |
| | | (IOB value = 0.35) | | | | |
| | | Neopentyl glycol dicaprate | — | — | — | 79 |
| | | (IOB value = 0.25) | | | | |
| | | Glyceryl diisostearate | — | — | — | — |
| | | (IOB value = 0.29) | | | | |
| | | Diisostearyl malate | — | — | — | — |
| | | (IOB value = 0.28) | | | | |
| | Highly | PPG-3 dipivalate | — | — | — | — |
| | polar oil | (IOB value = 0.52) | | | | |
| | Hydrocarbon | Hydrogenated polydecene | — | — | — | — |
| | oil | (IOB value = 0) | | | | |
| Total (%) | | | 100 | 100 | 100 | 100 |
| Apparent IOB value of component (C) | | | 0.26 | 0.26 | 0.35 | 0.25 |
| Content of component (B)/content of component (A) | | | 0 | 0.083 | 0.083 | 0.083 |
| Lightness difference (ΔL) | | | Reference | 1.79 L | 0.62 L | 2.38 L |
| | | | color | | | |
| | | | Example | Example | Comparative | Comparative |
| Constituent component | | | 7 | 8 | Example 3 | Example 4 |
| Additional component | | Polyethylene/ | 8.0 | 8.0 | 8.0 | 8.0 |
| | | microcrystalline wax | | | | |
| Component (A) | | Yellow iron oxide treated with | 12 | 12 | 12 | 12 |
| | | isostearyl sebacate, | | | | |
| | | disodium stearoyl glutamate, | | | | |
| | | and aluminum hydroxide | | | | |
| Component (B) | | (Polyglyceryl-2 isostearate/ | 1.0 | 1.0 | 1.0 | 1.0 |
| | | dimer dilinoleate) copolymer | | | | |
| Component | Polar oil | Polyglyceryl-2 triisostearate | — | — | — | — |
| (C) | | (IOB value = 0.26) | | | | |
| | | Pentaerythrityl | — | — | — | — |
| | | tetraethylhexanoate | | | | |
| | | (IOB value = 0.35) | | | | |
| | | Neopentyl glycol dicaprate | — | — | — | — |
| | | (IOB value = 0.25) | | | | |
| | | Glyceryl diisostearate | 79 | — | — | — |
| | | (IOB value = 0.29) | | | | |
| | | Diisostearyl malate | — | 79 | — | — |
| | | (IOB value = 0.28) | | | | |
| | Highly | PPG-3 dipivalate | — | — | 79 | — |
| | polar oil | (IOB value = 0.52) | | | | |
| | Hydrocarbon | Hydrogenated polydecene | — | — | — | 79 |
| | oil | (IOB value = 0) | | | | |

| | | | | |
|---|---|---|---|---|
| Total (%) | 100 | 100 | 100 | 100 |
| Apparent IOB value of component (C) | 0.29 | 0.28 | 0.51 | 0 |
| Content of component (B)/content of component (A) | 0.083 | 0.083 | 0.083 | 0.083 |
| Lightness difference (ΔL) | 1.42 L | 1.16 L | −0.57 D* | −4.96 D* |

*Reference color was set by removing the dispersant from the Comparative Example.

As is clear from the results in Table 2, it was found that the compositions of Examples 4 to 8 all had improved dispersibility of the component (A) compared to Comparative Example 2.

In the cases of Comparative Examples 3 and 4, it was found that dispersibility of the component (A) could not be improved by each of the formulations.

### Examples 9 and 10 and Comparative Examples 5 and 6

Based on the formulations in Table 3, the compositions of Examples 9 and 10 and Comparative Examples 5 and 6 were each prepared. The lightness value (L value) of each of the prepared compositions was measured, and the lightness difference from a reference color was determined. Results are shown in Table 3. Note that Comparative Example 5 was used as the reference color.

TABLE 3
(Table 3 Examples 9 and 10 and Comparative Examples 5 and 6)

| | | | Comparative | Example | Example | Comparative |
|---|---|---|---|---|---|---|
| Constituent component | | | Example 5 | 9 | 10 | Example 6 |
| Additional component | | Polyethylene/microcrystalline wax | 8.0 | 8.0 | 8.0 | 8.0 |
| Component (A) | | Yellow iron oxide treated with isostearyl | 12 | 12 | 12 | 12 |
| | | sebacate, disodium stearoyl glutamate, and | | | | |
| | | aluminum hydroxide | | | | |
| Component (B) | | (Polyglycery1-2 isostearate/dimer | — | 1.0 | 1.0 | 1.0 |
| | | dilinoleate) copolymer | | | | |
| Component | Polar oil | Polyglyceryl-2 triisostearate | 80 | 61 | 43 | 25 |
| (C) | | (IOB value = 0.26) | | | | |
| | Highly | PPG-3 dipivalate | — | 18 | 36 | 54 |
| | polar oil | (IOB value = 0.52) | | | | |

| | | | | |
|---|---|---|---|---|
| Total | 100 | 100 | 100 | 100 |
| Apparent IOB value of component (C) | 0.26 | 0.32 | 0.37 | 0.43 |
| Content of component (B)/content of component (A) | 0 | 0.083 | 0.083 | 0.083 |
| Lightness difference (ΔL) | Reference | 0.31 L | 0.35 L | −0.50 D |
| | color | | | |

As is clear from the results in Table 3, it was found that the compositions of Examples 9 and 10 both had improved dispersibility of the component (A) compared to Comparative Example 5. At the same time, it was found that the composition of Comparative Example 6 had inferior dispersibility of the component (A) compared to Comparative Example 5.

### Examples 11 and 12 and Comparative Examples 7 and 8

Based on the formulations in Table 4, the compositions of Examples 11 and 12 and Comparative Examples 7 and 8 were each prepared. The lightness value (L value) of each of the prepared compositions was measured, and the lightness difference from a reference color was determined. Results are shown in Table 4. Note that Comparative Example 7 was used as the reference color.

TABLE 4
(Table 4 Examples 11 and 12 and Comparative Examples 7 and 8)

| | | Comparative | Example | Example | Comparative |
|---|---|---|---|---|---|
| Constituent component | | Example 7 | 11 | 12 | Example 8 |
| Additional | Polyethylene/microcrystalline | 8.0 | 8.0 | 8.0 | 8.0 |
| component | wax | | | | |
| Component | Yellow iron oxide treated | 12 | 12 | 12 | 12 |
| (A) | with isostearyl sebacate, | | | | |
| | disodium stearoyl glutamate, | | | | |
| | and aluminum hydroxide | | | | |
| Component | (Polyglyceryl-2 isostearate/ | — | 1.0 | 1.0 | 1.0 |
| (B) | dimer dilinoleate) copolymer | | | | |
| Component | Triisostearin [polar oil] | 80 | 59.5 | 49.5 | 39.5 |
| | (IOB value = 0.16) | | | | |
| (C) | Hydrogenated polydecene | — | 19.5 | 29.5 | 39.5 |
| | [hydrocarbon oil] | | | | |
| | (IOB value = 0) | | | | |

| | | | | |
|---|---|---|---|---|
| Total | 100 | 100 | 100 | 100 |
| Apparent IOB value of component (C) | 0.16 | 0.12 | 0.099 | 0.079 |
| Content of component (B)/ | 0 | 0.083 | 0.083 | 0.083 |
| content of component (A) | | | | |
| Lightness difference (ΔL) | Reference | 0.70 L | 0.67 L | −1.27 D |
| | color | | | |

As is clear from the results in Table 4, it was found that the compositions of Examples 11 and 12 both had improved dispersibility of the component (A) compared to Comparative Example 7. At the same time, it was found that the composition of Comparative Example 8 had inferior dispersibility of the component (A) compared to Comparative Example 7.

### Examples 13 and 14 and Comparative Examples 9 to 11

Based on the formulations in Table 5, the compositions of Examples 13 and 14 and Comparative Examples 9 to 11 were each prepared. The lightness value (L value) of each of the prepared compositions was measured, and the lightness difference from a reference color was determined. Results are shown in Table 5. Note that for Comparative Example 10, Comparative Example 9 was used as the reference color, and for Examples 13 and 14, Comparative Example 11 was used as the reference color.

TABLE 5
(Table 5 Examples 13 and 14 and Comparative Examples 9 to 11)

| | | Comparative | Comparative | Comparative | Example | Example |
|---|---|---|---|---|---|---|
| Constituent component | | Example 9 | Example 10 | Example 11 | 13 | 14 |
| Additional | Polyethylene/microcrystalline wax | 8.0 | 8.0 | 8.0 | 8.0 | 8.0 |
| component | | | | | | |
| Component | Yellow iron oxide treated with isosteary! | — | — | 12 | 12 | 12 |
| (A) | sebacate, disodium stearoyl glutamate, | | | | | |
| | and aluminum hydroxide | | | | | |
| Component | Yellow iron oxide treated with silicone | 12 | 12 | — | — | — |
| (A′) | | | | | | |
| Component | (Polyglyceryl-2 isostearate/dimer | — | 1.0 | — | 12 | 12 |
| (B) | dilinoleate) copolymer | | | | | |
| Component | Polyglycery1-2 triisostearate [polar oil] | 80 | 79 | 70 | 58 | 48 |
| (C) | (IOB value = 0.26) | | | | | |
| | Diphenylsiloxy phenyltrimethicone | — | — | 10 | 10 | 20 |
| | [silicone oil] | | | | | |

| | | | | | |
|---|---|---|---|---|---|
| Total | 100 | 100 | 100 | 100 | 100 |
| Apparent IOB value of component (C) | 0.26 | 0.26 | 0.26 | 0.26 | 0.26 |
| Content of component (B)/content of component (A) | — | — | 0 | 1 | 1 |
| Lightness difference (ΔL) | Reference | −0.36 D | Reference | 2.15 L | 1.98 L |
| | color | | color | | |

As is clear from the results in Table 5, it was found that the compositions of Examples 13 and 14 both had improved dispersibility of the component (A) compared to Comparative Example 11. At the same time, it was found that the composition of Comparative Example 10 had inferior dispersibility of the component (A) compared to Comparative Example 9.

### Examples 15 to 17 and Comparative Examples 12 to 14

Based on the formulations in Table 6, the compositions of Examples 15 to 17 and Comparative Examples 12 to 14 were each prepared. The lightness value (L value) of each of the prepared compositions was measured, and the lightness difference from a reference color was determined. Results are shown in Table 6. Note that for Examples 15 to 17, Comparative Examples 12 to 14 were used, respectively, as the reference colors.

TABLE 6
(Table 6 Examples 15 to 17 and Comparative Examples 12 to 14)

| | | Comparative | | Comparative | Example | Comparative | Example |
|---|---|---|---|---|---|---|---|
| Constituent component | | Example 12 | Example 15 | Example 13 | 16 | Example 14 | 17 |
| Additional | Polyethylene/microcrystalline wax | 8.0 | 8.0 | 8.0 | 8.0 | 8.0 | 8.0 |
| component | | | | | | | |
| Component | Yellow iron oxide treated with sodium dilauroyl | 12 | 12 | — | — | — | — |
| (A) | glutamate lysine and magnesium chloride | | | | | | |
| | Red iron oxide treated with disodium stearoyl | — | — | 12 | 12 | — | — |
| | glutamate and aluminum hydroxide | | | | | | |
| | Yellow iron oxide treated with magnesium stearate | — | — | — | — | 12 | 12 |
| Component | (Polyglyceryl-2 isostearate/dimer dilinoleate) | — | 1.0 | — | 1.0 | — | 1.0 |
| (B) | copolymer | | | | | | |
| Component | Polyglyceryl-2 triisostearate [polar oil] | 80 | 79 | 80 | 79 | 80 | 79 |
| (C) | (IOB value = 0.26) | | | | | | |

| | | | | | | |
|---|---|---|---|---|---|---|
| Total | 100 | 100 | 100 | 100 | 100 | 100 |
| Apparent IOB value of component (C) | 0.26 | 0.26 | 0.26 | 0.26 | 0.26 | 0.26 |
| Content of component (B)/content of component (A) | 0 | 0.083 | 0 | 0.083 | 0 | 0.083 |
| Lightness difference (ΔL) | Reference | 0.23 L | Reference | 0.09 L | Reference | 0.15 L |
| | color | | color | | color | |

As is clear from the results in Table 6, it was found that the compositions of Examples 15 to 17 had improved dispersibility of the component (A) compared to Comparative Examples 12 to 14, respectively.

### Example 18 and Comparative Example 15

Based on the formulations in Table 7, the compositions of Example 18 and Comparative Example 15 were each prepared. The lightness value (L value) of each of the prepared compositions was measured, and the lightness difference from a reference color was determined. Results are shown in Table 7. Note that Comparative Example 15 was used as the reference color.

TABLE 7
(Table 7 Example 18 and Comparative Example 15)

| | | Comparative | Example |
|---|---|---|---|
| Constituent component | | Example 15 | 18 |
| Component | Yellow iron oxide treated | 27.2 | 27.2 |
| (A) | with isostearyl sebacate, | | |
| | disodium stearoyl glutamate, | | |
| | and aluminum hydroxide | | |
| Component | (Polyglyceryl-2 isostearate/ | — | 9.1 |
| (B) | dimer dilinoleate) copolymer | | |
| Component | Triisostearin [polar oil] | 66.4 | 57.3 |
| (C) | (IOB value = 0.16) | | |
| | Trimethylolpropane | 6.4 | 6.4 |
| | triethylhexanoate [polar oil] | | |
| | (IOB value = 0.33) | | |

| | | |
|---|---|---|
| Total | 100 | 100 |
| Apparent IOB value of component (C) | 0.17 | 0.18 |
| Content of component (B)/ | 0 | 0.33 |
| content of component (A) | | |
| Lightness difference (ΔL) | Reference color | 2.19 L |

As is clear from the results in Table 7, it was found that the composition of Example 18 had improved dispersibility of the component (A) compared to Comparative Example 15.

1. A composition that is a cosmetic composition, comprising
a component (A): an inorganic pigment surface-treated with (i) or (ii) below:
(i) an amide compound obtained by dehydration condensation of an acidic amino acid and a carboxylic acid, and/or a salt thereof, or
(ii) a metal soap;
a component (B): a (polyglyceryl-2 isostearate/dimer dilinoleate) copolymer; and
a component (C): a liquid oil comprising a polar oil,
wherein an apparent IOB value of the component (C) is 0.09 or greater and 0.37 or less.

2. The composition according to claim 1, wherein with respect to the (i), number of carbon atoms in the carboxylic acid is 18 or less, and/or,
wherein with respect to the (ii), number of carbon atoms in a fatty acid forming the metal soap is 12 or greater.

3. The composition according to claim 1, wherein an IOB value of the polar oil is 0.10 or greater and 0.55 or less.

4. The composition according to claim 1, wherein a content of the component (B) is 24% by mass or less.

5. The composition according to claim 1, wherein a ratio of content of the component (B) to a content of the component (A) (a content of the component (B)/a content of the component (A)) is 0.08 or greater and 2.0 or less.

6. The composition according to claim 1, wherein, when further comprising a silicone oil, a content of the silicone oil is 50% by mass or less, and the apparent IOB value of the component (C) is calculated by excluding the silicone oil.

7. The composition according to claim 1, wherein the polar oil comprises at least one selected from a group consisting of triisostearin, polyglyceryl-2 triisostearate, pentaerythrityl tetraethylhexanoate, neopentyl glycol dicaprate, glyceryl diisostearate, diisostearyl malate, and trimethylolpropane triethylhexanoate.

8. The composition according to claim 1, wherein a content of the polar oil is 50% by mass or greater.
